(19) United States
(12) Patent Application Publication
(10) Pub. No.: US 2008/0045909 A1
(43) Pub. Date: Feb. 21, 2008

(54) Topical Delivery of a Nitric Oxide Donor to Improve and Skin Appearance

(75) Inventor: Eric Thor Fossel, Cambridge, MA (US)

Correspondence Address: WOLF GREENFIELD & SACKS, P.C., 600 ATLANTIC AVENUE, BOSTON, MA, 02210-2206, US

(73) Assignee: STRATEGIC SCIENCE & TECHNOLOGIES, LLC., CAMBRIDGE, MA (US)

(21) Appl. No.: 10/590,300

(22) PCT Filed: Feb. 23, 2005
(86) PCT No.: PCT/US05/05726

§ 371 Date: Jun. 21, 2007

Related U.S. Application Data
(60) Provisional application No. 60/546,214, filed on Feb. 23, 2004.
(60) Provisional application No. 60/563,566, filed on Apr. 19, 2004.

(51) Int. Cl. A61M 35/00

(52) U.S. Cl. 604/290; 424/401

(57) ABSTRACT

This invention generally relates to improvement of the body and skin appearance, for example enhancing the appearance of sagging, wrinkled, or cellulite-afflicted areas of the skin and body, through the local delivery of a nitric oxide donor, for example, using delivery vehicles such as lotions, creams, liquids, and/or transdermal patches. In some embodiments, a delivery vehicle containing a nitric oxide donor, for example, L-arginine (an important biological precursor) or its derivatives in a sufficient concentration to improve the appearance of a selected area of the body may be applied. In certain cases, one or more agents may also be included that aid in the transfer of the nitric oxide donor into the tissue, which may overcome the resistance to transfer into the skin. Non-limiting examples of suitable agents include agents able to create hostile biophysical environments, for instance, choline chloride, magnesium chloride, and/or sodium chloride.

### RELATED APPLICATIONS

This application claims the benefit of U.S. Provisional Patent Application Ser. No. 60/546,214, filed Feb. 23, 2004, entitled “Topical Delivery of a Nitric Oxide Donor to Improve Body and Skin Appearance,” by E. T. Fossel; and U.S. Provisional Patent Application Ser. No. 60/563,566, filed Apr. 19, 2004, entitled “Transdermal Delivery of L-Arginine for the Purpose of Enhancing the Appearance of the Female Breast,” by E. T. Fossel. Each of the above applications is incorporated herein by reference.

### FIELD OF INVENTION

This invention generally relates to methods and compositions for improving body and skin appearance.

### BACKGROUND

There have been many approaches to improving body and skin appearance, using both systemic and topical approaches. One method of tightening skin to improve appearance is through the use of cosmetic surgery. For instance, for sagging skin or larger wrinkles for double chins, an individual may resort to cosmetic surgery, for example, a facelift or a tuck. Sagging breasts have also been treated surgically. However, the problems associated with this approach are obvious with the high cost and the risks associated with undergoing any medically unnecessary surgery.

Another surgical method to improve skin appearance of areas of the skin, such as the chins and arms, is through liposuction. Liposuction is effective for improving the appearance of skin, but it has a very high cost and there can be side effects, such as infections that can lead to death.

Radiofrequency energy is another method increasingly being used to tighten skin without the need for surgery, such as in a conventional facelift, reducing some of the potential surgical risks such as infection and anesthesia. This medical procedure is still troublesome to many individuals, however, because it can cause damage to underlying tissues.

Treatments of the skin without the use of cosmetic surgery include many different techniques, but there are relatively few treatments that are effective in providing any noticeable benefits relative to the prohibitive costs. For instance, a popular method of providing the appearance of a tightening of the skin is to remove small wrinkles through the use of alpha lipoic acid. This treatment does not cause much of a tightening effect, only the removal of time wrinkles, thereby providing the appearance of tightening. Although many treatments can produce a negative reaction in some people, adverse reactions to lipoic acid are somewhat less common than to agents such as Retin-A, vitamin C or glycolic acid. Alpha lipoic acid is useful in treating small wrinkles but it can result in rashes, because of reaction to the acid.

### SUMMARY OF THE INVENTION

This invention generally relates to improvement of the body and skin appearance. The subject matter of the present invention involves, in some cases, interrelated products, alternative solutions to a particular problem, and/or a plurality of different uses of one or more systems and/or articles.

The instant invention provides, in one aspect, beneficial effects in the appearance of the body and skin, for instance by smoothing skin that is wrinkled, sagging, or cellulite-afflicted. In one set of embodiments, the application of a delivery vehicle such as a cream, liquid, lotion, spray, aerosol, or transdermal patch containing nitric oxide and/or a nitric oxide donor in a sufficient concentration to improve the appearance of a selected area of the body may be applied. As discussed in more detail herein, the nitric oxide donor is at least one compound that donates, transfers, and/or releases nitric oxide, elevates endogenous levels of endothelium-derived relaxing factor, stimulates endogenous synthesis of nitric oxide, and/or is a substrate for nitric oxide synthase.

As examples, a cream may be used to treat the appearance of double chins, crows feet, and/or a multitude of other cosmetic problems associated with wrinkled, sagging, and/or dimpled appearance that affect the smoothness of the surface of the skin. The appearance of sagging skin that may occur on areas of the body such as the breasts, arms, legs, back, ankles, stomach, “love handles,” and/or buttocks can be treated using embodiments of the invention to produce a more desirable appearance by applying a delivery vehicle such as a cream, liquid, lotion, spray, aerosol, or transdermal patch containing nitric oxide and/or a nitric oxide donor. For example, a person with breast ptosis (e.g., pseudoptosis, partial ptosis, or true ptosis) may be treated using an embodiment of the invention. In one embodiment, the nitric oxide donor includes L-arginine or its derivatives in a quantity sufficient to produce the desired cosmetic effects. Other embodiments are more fully described herein.

In another embodiment of the invention, a cream containing a nitric oxide donor (e.g., L-arginine or its derivatives) at an effective concentration, may create a hostile biophysical environment that facilitates absorption of the nitric oxide donor into the skin. In some cases, an agent or agents may be combined with a sufficient concentration of nitric oxide donor to create the hostile biophysical environment. In other cases, the nitric oxide donor may be sufficient to create the hostile biophysical environment.

In some cases, the instant invention may be used to enhance the appearance of the body using the body's natural mechanisms. For example, the instant invention may be used to remove small wrinkles, to remove the appearance of the condition commonly known as a “double chin,” to tighten sagging breasts, to smooth cellulite-afflicted skin, to smooth facial tissue without surgery, to lift sagging arm tissue, to lift and tighten sagging buttocks, or to lift and tighten sagging leg skin. Additional details and applications are provided below.

Thus, in one aspect, the method includes an act of applying a delivery vehicle comprising a nitric oxide donor to a region of sagging skin for a period of time sufficient to reduce sagging. The method, according to another aspect, includes an act of applying a delivery vehicle to a region of skin containing a nitric oxide donor for a period of time sufficient to allow the skin to absorb a sufficient quantity of nitric oxide to produce a smoother surface in the region of skin. In yet another aspect, the method includes an act of administering, to a subject diagnosed as having breast ptosis, a composition comprising a nitric oxide donor.

In another aspect, the method includes a use of a composition in the manufacture of a medicament for treatment of sagging skin, where the composition comprises a nitric oxide donor. In yet another aspect, the method includes a use of a composition in the manufacture of a medicament for producing a smoother surface in a region of skin, where the composition comprises a nitric oxide donor. In still another aspect, the method includes a use of a composition in the manufacture of a medicament for the treatment of breast ptosis, where the composition comprises a nitric oxide donor.

The present invention, in another aspect, is directed to a method of making one or more of the embodiments described herein. In yet another aspect, the present invention is directed to a method of using one or more of the embodiments described herein. In still another aspect, the present invention is directed to a method of promoting one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures. In the figures:

FIGS. 1A-1B illustrate the use of an embodiment of the invention to treat a person's breasts; and

FIGS. 2A-2B illustrate the use of another embodiment of the invention in the treatment of a person's breasts.

### DETAILED DESCRIPTION

This invention generally relates to improvement of the body and skin appearance, for example enhancing the appearance of sagging, wrinkled, or cellulite-afflicted areas of the skin and body, through the local delivery of a nitric oxide donor, for example, using delivery vehicles such as lotions, creams, liquids, sprays, aerosols, and/or transdermal patches. In some embodiments, a delivery vehicle containing a nitric oxide donor, for example, L-arginine (an important biological precursor) or its derivatives in a sufficient concentration to improve the appearance of a selected area of the body may be applied. In certain cases, one or more agents may also be included that aid in the transfer of the nitric oxide donor into the tissue, which may overcome the resistance to transfer into the skin. Non-limiting examples of suitable agents include agents able to create hostile biophysical environments, for instance, choline chloride, magnesium chloride, and/or sodium chloride.

In one aspect, the topical application of nitric oxide and/or a nitric oxide donor (for example, L-arginine) may be used to cause a beneficial effect to the area of the skin applied, for example, a cosmetic effect, such as improving body or skin appearance. The nitric oxide may cause changes in the skin through natural biological responses that react to the presence of nitric oxide. For example, in response to the presence of nitric oxide, the skin may smoothen, tighten, or become more firm (i.e., the viscoelasticity of the skin may increase), which may result in an improvement in appearance. In some cases, skin having a sagging appearance may be treated using various embodiments of the invention. For example, the sagging appearance of the skin and certain body parts such as breasts may be reduced; for instance, a breast may appear fuller and/or raised after treatment. As a particular example, a person with breast ptosis (e.g., pseudoptosis, partial ptosis, or true ptosis) may be treated using an embodiment of the invention. Other non-limiting examples include, but are not limited to, treatment of the arms, legs, back, ankles, stomach, “love handles,” and/or buttocks. In some cases, the topical application of nitric oxide and/or a nitric oxide donor may improve appearance by causing an increase in tissue volume, which may result in increased size or firmness, and/or decrease sagging. In certain instances, the improved appearance may be measured, for example, by measuring a change in the viscoelasticity of the skin.

In some embodiments, nitric oxide and/or a nitric oxide donor may be administered using a delivery vehicle such as a cream, liquid, lotion, spray, aerosol, or transdermal patch. Examples of delivery vehicles are discussed below. The delivery vehicle may promote transfer into the skin of an effective concentration of nitric oxide, directly or indirectly, through a nitric oxide donor capable of penetrating into at least a portion of the skin. For instance, the delivery vehicle may include one or more penetrating agents, as further described herein. In some embodiments, the delivery vehicle may include a hostile biophysical environment, e.g., using a penetrating agent, and/or using the nitric oxide and/or nitric oxide donor, alone or in combination with other agents, as further discussed herein.

In certain embodiments of the invention, multiple treatments of the delivery vehicle may increase the duration of the effects of nitric oxide, for example two, three, four, five, or more treatments may be applied, depending on the particular application. For example, with repeated administrations, the beneficial effects of each treatment may be extended up to ten or twenty hours after treatment, or more in some cases. In certain cases, the concentration of nitric oxide and/or a nitric oxide donor can be reduced after the initial treatment to maintain the same desired duration of cosmetic effect. Such treatments may be given at any suitable frequency, depending on the particular application, for example, every 4 hours, every 8 hours, every 12 hours, every 18 hours, every 1 day, every 2 days, every 3 days, every week, etc. For instance, the treatment may be provided between about 2 and about 30 times within a time period of about 30 days. In some cases, the first treatment may be given at a higher level or concentration than subsequent treatments.

A “nitric oxide donor,” as used herein, is a compound that contains a nitric oxide moiety, where the compound is able to release nitric oxide and/or chemically transfer the nitric oxide moiety to another molecule, directly or indirectly, for example, through a biological process. The nitric oxide donor may release nitric oxide into the skin, and/or tissues such as muscles and/or elements of the circulatory system in close proximity to the surface of the skin. Non-limiting examples of nitric oxide donors include arginine (e.g., L-arginine and/or D-arginine), arginine derivatives (e.g., L-arginine hydrochloride and/or D-arginine hydrochloride), nitroglycerin, polysaccharide-bound nitric oxide-nucleophile adducts, N-nitroso-N-substituted hydroxylamines, 1,3-(nitrooxymethyl)phenyl-2-hydroxybenzoate, etc., as described in more detail herein. In some cases, the concentration of nitric oxide and/or the nitric oxide donor may be tailored to have a duration of effective treatment of at least about 3 hours, at least about 5 hours, or at least about 8 hours or more in certain instances. The duration may also be controlled, for instance, by controlling the concentration of a penetrating agent used in conjunction with nitric oxide and/or the nitric oxide donor. The actual concentration for a particular application can be determined by those of ordinary skill in the art using no more than routine experimentation, for example, by measuring the amount of transport of nitric oxide and/or the nitric oxide donor as a function of concentration in vitro across cadaver skin or suitable animal models, skin grafts, synthetic model membranes, or the like.

As a particular non-limiting example, in one embodiment, nitric oxide is provided using L-arginine, for example, at a concentration of at least about 0.50% by weight (wt % or w/v) of L-arginine (optionally with one or more penetrating agents as discussed herein, for example, a penetrating agent able to create a hostile biophysical environment), at least about 0.75 wt %, at least about 1 wt %, at least about 2 wt %, at least about 3 wt %, at least about 5 wt %, at least about 7 wt %, at least about 10 wt %, or at least about 15 wt %. The L-arginine may be present in a suitable delivery vehicle, such as a cream or a lotion. L-arginine may be particularly useful in some cases due to its low toxicity, its high solubility, or its low cost.

Nitric oxide and/or a nitric oxide donor may optionally be combined with an agent or environment to aid in penetration. Examples include, but are not limited to, high ionic strength environments, agents or environments able to neutralize charge in a complex, and/or liposomes or other biological carriers, as discussed herein. In some embodiments, a hostile biophysical environment may be used, as further discussed herein. For example, a delivery vehicle (for example, a cream) containing nitric oxide and/or a nitric oxide donor may be provided at a concentration sufficient to produce a hostile biophysical environment, which may allow a sufficient amount of nitric oxide and/or a nitric oxide donor to produce a desired effect. In one embodiment, a hostile biophysical environment may be created using ionic salts, which may be at high concentrations in some cases. Examples include sodium chloride, magnesium chloride, calcium chloride, and/or choline chloride. In some cases, the ionic salt(s) is at a concentration sufficient to aid in tissue absorption of nitric oxide and/or a nitric oxide donor. As another example, nitric oxide and/or a nitric oxide donor may also be used in conjunction with an adjunct, such as theophylline. As yet another example, a nitric oxide and/or a nitric oxide donor may itself be at a concentration within the delivery vehicle sufficient to create a hostile biophysical environment. Other examples of penetrating agents include, but are not limited to, cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, glycerol); sulfoxides (e.g., dimethylsulfoxide); or terpenes (e.g., cyclohexene).

In another embodiment, a nitric oxide donor can include polysaccharide-bound nitric oxide-nucleophile adduct, such as those described in U.S. Pat. No. 5,691,423, the contents of which are incorporated herein by reference. Thus, in some cases, a polymeric composition capable of releasing nitric oxide may include a nitric oxide releasing N₂O₂-functional group bound to a polymer. In some cases, the polymeric composition comprise a polysaccharide. The polymeric composition may release NO in a controlled manner for effective dosing. The nitric oxide donor may also be a chitosan-based polymer in some cases, for example, as described in U.S. Pat. No. 6,451,337, the contents of which are incorporated herein by reference. In certain instances, any of the above-described polymeric composition may be lipophilic, biodegradable, and/or biocompatable. For instance, in some cases, the polymeric composition may degrade into naturally occurring products.

In yet another embodiment, a nitric oxide donor can include N-nitroso-N-substituted hydroxylamines for use as nitric oxide donors. Examples of N-nitroso-N-substituted hydroxylamines include those described in U.S. Pat. No. 5,698,738, the contents of which are incorporated by reference herein. In some cases, the nitric oxide donor is a NONOate anion linked to an ortho-substituted aryl, a heteroaromatic substituent, asteroid, or a catecholamine. Examples of ortho substituents include alkoxy, halo, or alkyl. If the hydroxylamine is part of a salt, the counter-ion may be an alkali metal, an alkaline-earth metal, or an ammonium or substituted ammonium group. Non-limiting examples of nitric oxide donors include N-nitroso-N-(1-naphthyl)-hydroxylamine (ammonium or sodium salt), N-nitroso-N-(2-methylphenyl)-hydroxylamine (salt), N-nitroso-N-(2-methoxyphenyl)-hydroxylamine (salt), N-nitroso-N-(2-ethylphenyl)-hydroxylamine (salt), N-nitroso-N-(2-isopropylphenyl)-hydroxylamine (salt), N-nitroso-N-(2,4-difluorophenyl)-hydroxylamine (salt), N-nitroso-N-(2,5-difluorophenyl)-hydroxylamine (salt), N-nitroso-N-(2-chlorophenyl)-hydroxylamine (salt), N-nitroso-N-(2,3-dichlorophenyl)-hydroxylamine (salt), N-nitroso-N-(2,4-dichlorophenyl)-hydroxylamine (salt), N-nitroso-N-(2,5-dichlorophenyl)-hydroxylamine (salt), N-nitroso-N-(2-bromophenyl)-hydroxylamine (salt), N-nitroso-N-(5-fluoro-2-methylphenyl)-hydroxylamine (salt), N-nitroso-N-(4-fluoro-2-methylphenyl)-hydroxylamine (salt), N-nitroso-N-(4-choro-2-methylphenyl)-hydroxylamine (salt), or N-nitroso-N-(3-choro-2-methylphenyl)-hydroxylamine (salt).

In still another embodiment, the nitric oxide donor includes a compound containing a sulflhydryl group and a NO donor group. The compound may contain an acetylated sulflhydryl group linked to an aromatic ring or a heteroaromatic ring with a nitrogen in the ring structure, which ring may be substituted in some cases by a substituent bearing a terminal —ONO. Examples of such compounds include those described in U.S. Pat. No. 6,642,260, the contents of which are incorporated herein by reference. Examples of nitric oxide donors include, but are not limited to, trans-1,2-dinitrato-4,5-dithiane; 2,2′-dithiodiethanol-dinitrate; 1,1-diemethanol-dinitrate-3,4-dithiane; 1,1′-bisthiomethyl-3,4-dihydroxy-cyclohexane-dinitrate ester; thiotyl alcohol nitrite ester; or 1,2-dihydroxy-dinitrate-6,8-dithiane.

In another embodiment, the nitric oxide donor includes 1,3-(nitrooxymethyl)phenyl-2-hydroxybenzoate and other related compounds, e.g., as described in U.S. Pat. No. 6,538,033, the contents of which are incorporated by reference herein.

In still another embodiment, a nitric oxide donor is provided by topically applying a first gel comprising a nitrite salt and a biocompatible reductant, and a second gel comprising an acid. Examples of such topical administration include those described in U.S. Pat. No. 6,103,275, herein incorporated by reference. In some cases, the acid can have a pKa between about 1 and about 4.

The nitric oxide donor, in yet other embodiments, is a nitroxide. Examples of nitroxides include, but are not limited to, sodium nitroprusside (Nipride), S-nitrosoacetylpenacil-lamine (SNAP), 3-morpholino-synoniminhydrochloride (SIN-1), 3-morpholino-N-athoxycarbonly-syndnonimin (molsidomin), amyl nitrite (isoamyl nitrite), nitroglycerin (glyceryl trinitrite), isosorbide dinitrate (Isodil), isosorbide-5-monoitrite (Imur), or erythrityl tetranitrate (cardilate). Additional examples can be found in U.S. Pat. No. 6,617,337 herein incorporated by reference.

In another embodiment, the nitric oxide donor is a nitric oxide-releasing amidine- or enamine-derived diazeniumdiolate, for example, as taught in U.S. Pat. No. 6,511,991, the contents of which are incorporated herein by reference. In yet another embodiment, the nitric oxide donor is a piperidine or a pyrrolidine derivative. Examples of these compounds are described in U.S. Pat. No. 6,448,267, the contents of which are incorporated by reference herein.

As discussed, in some embodiments, nitric oxide and/or a nitric oxide donor may be contained in a delivery vehicle such as a cream, liquid, lotion, spray, aerosol, or transdermal patch (which may contain a cream, liquid, lotion, spray, aerosol, or other formulation that allows transport of nitric oxide and/or a nitric oxide donor to occur), optionally in combination with a penetrating agent. Those of ordinary skill in the art will know of systems and techniques for incorporating bioactive compounds within delivery vehicles such as a cream, liquid, lotion, spray, aerosol, or transdermal patch. For example, the concentration of nitric oxide and/or a nitric oxide donor (e.g., L-arginine or its derivatives) within a delivery vehicle such as a cream or lotion may be at least about 0.1% w/v, between about 0.1 to 25% w/v, between about 5% w/v and 25% w/v, between about 10% w/v and 25% w/v, etc. In some cases, the concentration of nitric oxide and/or a nitric oxide donor in the delivery vehicle can be reduced with the inclusion of a greater amount or concentration of penetrating agent, or increased to lengthen the beneficial effect.

Thus, as one particular example, a delivery vehicle such as a cream or lotion may contain a nitric oxide donor such as L-arginine hydrochloride with at least 12.5% weight by volume, combined with penetrating agents such as choline chloride having at least 10% weight by volume, sodium chloride with at least 5% weight by volume, and/or magnesium chloride with at least 5% weight by volume. In some cases, an adjunct such as theophylline may also be used (for example, at 10% weight by volume). If a cream is used, other materials may be present within the cream, for example, buffers, preservatives, surfactants, etc. For instance, the cream may include one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A and D, triethanolamine, methylparaben, aloe vera extract, imidazolidinyl urea, propylparaben, PND, or BHA.

As specific non-limiting examples, the cream may have one or more of (w/v): water (20-80%), white oil (3-18%), glyceryl stearate (0.25-12%), squalene (0.25-12%), cetyl alcohol (0.1-11%), propylene glycol stearate (0.1-11%), wheat germ oil (0.1-6%), polysorbate 60 (0.1-5%), propylene glycol (0.05-5%), collagen (0.05-5%), sorbitan stearate (0.05-5%), vitamin A (0.02-4%), vitamin D (0.02-4%), vitamin E (0.02-4%), triethanolamine (0.01-4%), methylparaben (0.01-4%), aloe vera extract (0/01-4%), imidazolidinyl urea (0.01-4%), propylparaben (0.01-4%), BHA (0.01-4%), L-arginine Hydrochloride (0.25-25%), sodium chloride (0.25-25%), magnesium chloride (0.25-25%), and/or choline chloride (0.25-25%). In this example, choline chloride, sodium chloride and magnesium chloride provide a high ionic strength environment for the highly charged molecule, L-arginine. This high ionic strength environment is an example of a hostile biophysical environment for L-arginine. That is, the highly charged ionic strength is an unfavorable environment for the highly charged L-arginine making the L-arginine anxious to move to a more hospitable, less charged environment such as human tissue. Hostile biophysical environments are discussed in more detail below.

Besides L-arginine and L-arginine hydrochloride, other non-limiting examples of nitric oxide donors include D,L-arginine, D-arginine, or alkyl (e.g., ethyl, methyl, propyl, isopropyl, butyl, isobutyl, tert-butyl, etc.) esters of L-arginine and/or D-arginine, and/or salts thereof, as well as other derivatives of arginine and other nitric oxide donors. For instance, non-limiting examples of pharmaceutically acceptable salts include hydrochloride, glutamate, butyrate, or glycolate (e.g., resulting in L-arginine glutamate, L-arginine butyrate, L-arginine glycolate, D-arginine hydrochloride, D-arginine glutamate, etc.). Other examples of nitric oxide donors include L-arginine-based compounds such as, but not limited to, L-homoarginine, N-hydroxy-L-arginine, nitrosylated L-arginine, nitrosylated L-arginine, nitrosylated N-hydroxy-L-arginine, nitrosylated N-hydroxy-L-arginine, citrulline, omithine, linsidomine, nipride, glutamine, etc., and salts thereof (e.g., hydrochloride, glutamate, butyrate, glycolate, etc.). Still other non-limiting examples of nitric oxide donors include S-nitrosothiols, nitrites, 2-hydroxy-2-nitrosohydrazines, or substrates of various forms of nitric oxide synthase. In some cases, the nitric oxide may be a compound that stimulates endogenous production of nitric oxide in vivo. Examples of such compounds include, but are not limited to, L-arginine, substrates of various forms of nitric oxide synthase, certain cytokines, adenosine, bradykinin, calreticulin, bisacodyl, phenolphthalein, OH-arginine, or endothelein.

A variety of methods for effecting or improving absorption of the active agent are also included in various embodiments of the invention. In some cases, a hostile biophysical environment may be used. In a hostile biophysical environment, the environment surrounding the nitric oxide and/or the nitric oxide donor (for example, L-arginine) may be such that the nitric oxide and/or nitric oxide donor is a chemically/energetically unfavorable environment, relative to the skin (i.e., the chemical potential of nitric oxide and/or the nitric oxide donor within the hostile biophysical environment is significantly greater than the chemical potential of nitric oxide and/or the nitric oxide donor within the skin, thus energetically favoring transport into the skin). In some cases, the delivery vehicle defines the biophysically hostile environment. In other cases, the nitric oxide and/or nitric oxide donor may be packaged in such a way that it is carried into tissue and/or its charge is neutralized by derivitization and/or by forming a neutral salt. Examples of biophysically hostile environments include, but are not limited to, high ionic strength environments (e.g., by the addition of ionic salts such as lithium chloride, sodium chloride, potassium chloride, calcium chloride, magnesium chloride, choline chloride, sodium fluoride, lithium bromide, etc., as well as combinations of these and/or other salts, for instance at high ionic strengths, such as between about 0.25 M and about 15 M, between about 5 M and about 15 M, between about 10 M and about 15 M, etc.); high or low pH environments (e.g., by adding pharmaceutically acceptable acids or bases, for example, such that the pH is between about 3 and about 7, between about 3 and about 6, between about 3 and about 5, between about 7 and about 11, between about 8 and about 11, between about 9 and about 11, etc.); or highly hydrophobic environments (e.g., by decreasing water content and increasing lipid, oil and/or wax content of the environment). Other highly charged molecules such as polylysine, polyglutamine, polyaspartate, etc., or copolymers of such highly charged amino acids may also be used in certain embodiments to create the hostile biophysical environment. Non-limiting examples of packaging which would be carried into tissue includes liposomes or emulsions of collagen, collagen peptides or other components of skin or basement membrane. Non-limiting examples of neutralization of charge include delivery of the nitric oxide and/or nitric oxide donor in the form or an ester or salt which is electronically neutral. For example, an arginine compound may be delivered as a neutral compound such as arginine glutamate.

A hostile biophysical environment may also be created in some embodiments by placing a nitric oxide donor that is relatively highly charged into a hydrophobic, oily environment such as in an oil-based cream or lotion containing little or no water. Absorption may further be aided by combining the use of hostile biophysical environments with the use of penetrating agents such as oleoresin capsicum or its constituents, or molecules containing heterocyclic rings to which are attached hydrocarbon chains.

The following are incorporated herein by reference: U.S. Provisional Patent Application Ser. No. 60/546,214, filed Feb. 23, 2004, entitled “Topical Delivery of a Nitric Oxide Donor to Improve Body and Skin Appearance,” by E. T. Fossel; U.S. Provisional Patent Application Ser. No. 60/563,566, filed Apr. 19, 2004, entitled “Transdermal Delivery of L-Arginine for the Purpose of Enhancing the Appearance of the Female Breast,” by E. T. Fossel; U.S. patent application Ser. No. 08/932,227, filed Sep. 17, 1997, entitled “Topical Delivery of Arginine of Cause Beneficial Effects,” by E. T. Fossel, published as 2002/0041903 on Apr. 11, 2002; U.S. patent application Ser. No. 10/201,635, filed Jul. 22, 2002, entitled “Topical Delivery of L-Arginine to Cause Beneficial Effects,” by E. T. Fossel, published as 2003/0028169 on Feb. 6, 2003; U.S. patent application Ser. No. 10/213,286, filed Aug. 5, 2002, entitled “Topical and Oral Arginine to Cause Beneficial Effects,” by E. T. Fossel, published as 2003/0018076 on Jan. 23, 2003; International Patent Application No. PCT/US98/19429, filed Sep. 17, 1998, entitled “A Delivery of Arginine to Cause Beneficial Effects,” by E. T. Fossel, published as WO 99/13717 on Mar. 25, 1999; U.S. Pat. No. 5,895,658, issued Apr. 20, 1999, entitled “Topical Delivery of L-Arginine to Cause Tissue Warming,” by E. T. Fossel; U.S. Pat. No. 5,922,332, issued Jul. 13, 1999, entitled “Topical Delivery of Arginine to Overcome Pain,” by E. T. Fossel; U.S. Pat. No. 6,207,713, issued Mar. 27, 2001, entitled “Topical and Oral Delivery of Arginine to Cause Beneficial Effects,” by E. T. Fossel; and U.S. Pat. No. 6,458,841, issued Oct. 1, 2002, entitled “Topical and Oral Delivery of Arginine to Cause Beneficial Effects,” by E. T. Fossel.

The following examples are intended to illustrate certain embodiments of the present invention, but do not exemplify the full scope of the invention.

### EXAMPLE 1

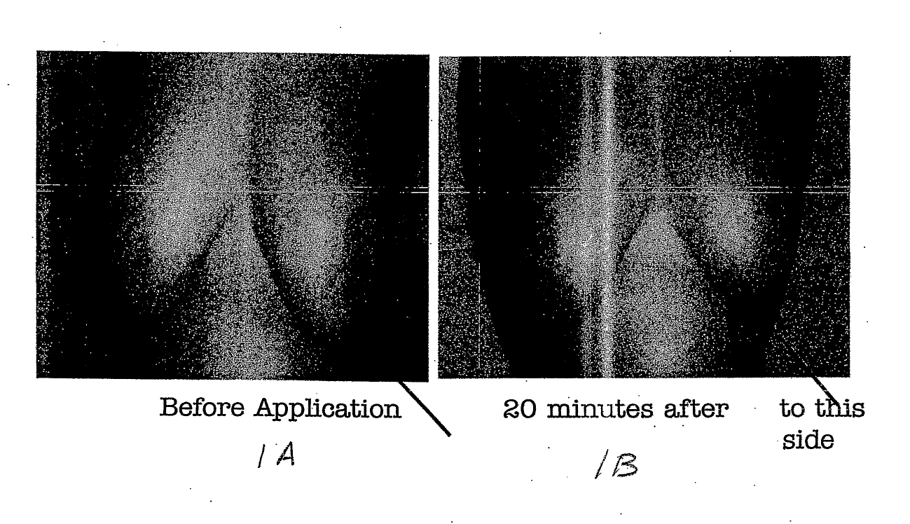

This example illustrates the reduction of breast sagging and an increase of breast firmness. In this example, a 60-year-old woman with pendulous breasts (FIG. 1A) was provided with a cream comprising L-arginine (12.5% w/v), sodium chloride (10% w/v), and magnesium chloride (5% w/v). The cream was applied to one of the breasts, which was rubbed in extensively for maximal absorption. After a period of approximately 20 minutes the treated breast was much fuller and raised up by about 1.5 inches (FIG. 1B). The effect of the initial treatment lasted for a period of about seven hours. The concentration of L-arginine could also be reduced to decrease the duration of the cosmetic effect of the initial application.

### EXAMPLE 2

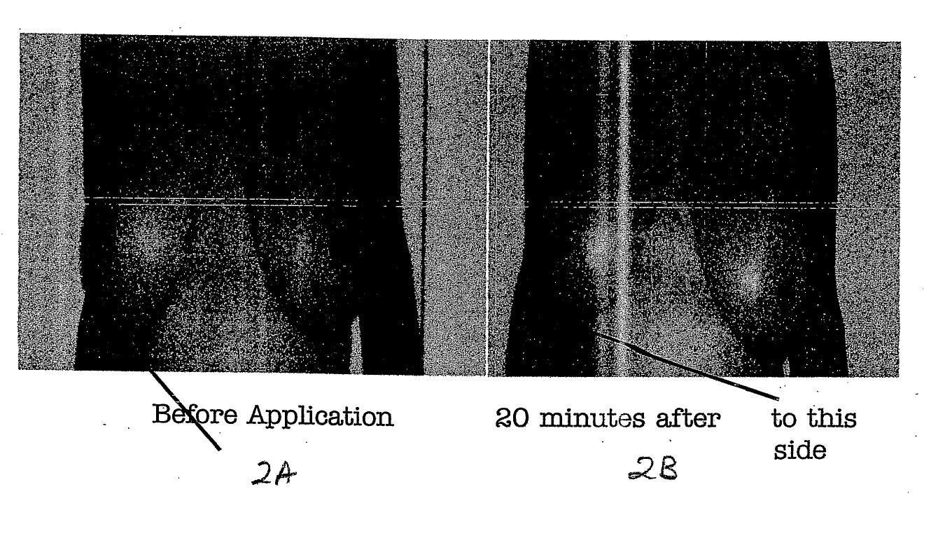

This example illustrates the reduction of breast sagging and an increase of breast firmness. In this example, a 47-year-old woman with pendulous breasts (FIG. 2A) applied a breast lifting cream comprising L-arginine (12.5% w/v), choline chloride (10% w/v), sodium chloride (10% w/v), and magnesium chloride (5% w/v). The breast lifting cream was rubbed vigorously into each breast for about five minutes. Within one hour both breasts were noticeably firmer and had been lifted about 2.75 inches (FIG. 2B). The effect of the initial treatment lasted for about five hours.

The treatment was continued daily for about a month. The lifting effect of the treatment had an effective duration of about 18 to 20 hours after about a month of daily use. The concentration of L-arginine could also be maintained to continue cosmetic benefits for up to twenty hours if the same cream is applied on a regular basis of once every 8 to 48 hours, or every 12 to 36 hours.

### EXAMPLE 3

In this example, an embodiment of the invention was used to improve the appearance of the neck and chin of a subject. A 59 year old woman with a large “double chin” applied a chin lifting cream comprising of a delivery vehicle of penetrating cream, L-arginine (12.5% w/v) sodium chloride (10% w/v), and magnesium chloride (5% w/v) to the tissue of her chin and under her chin by covering the area with the cream and rubbing it in for five minutes. After 15 minutes she looked in the mirror and observed that the “double chin” appeared to be completely gone and the skin on and under her chin was extremely smooth. The concentration of L-arginine could also be changed to lengthen or shorten duration of cosmetic benefits.

### EXAMPLE 4

This example illustrates the reduction of wrinkles in facial tissue. A 64 year old woman with extremely saggy wrinkled facial tissue applied a face lifting cream comprising of a delivery vehicle of penetrating cream, L-arginine (12.5% w/v), sodium chloride (10% w/v), and magnesium chloride (5% w/v) to her entire face (taking care to avoid the eyes) by completely covering the area with cream and rubbing it in for five minutes. Within 30 minutes she noticed the sagging facial tissue was substantially lifted and much smoother. The effect lasted for about 14 hours. She continued the treatment daily for two weeks, and at the end of the two weeks the treatment left her facial skin devoid of sagging tissue and it appeared to be completely smooth. The effect persisted for 14-16 hours.

### EXAMPLE 5

This example illustrates the lifting of sagging skin tissue in the buttocks. A 160 lb., 55 year old woman with flabby and sagging buttocks applied a buttock lifting cream comprising of a delivery vehicle of penetrating cream, L-arginine (12.5% w/v), sodium chloride (10% w/v), and magnesium chloride (5% w/v) to her buttocks by completely covering them with cream and rubbing the cream in for five minutes. She looked in the mirror in one hour and observed that the sagging was substantially reduced and that the buttocks were more firm. She continued the treatment daily for one month. At the end of the month her buttock sag was completely gone after application of the cream and they appeared to be firm and youthful. The effect persisted throughout the day.

### EXAMPLE 6

In this example, an embodiment of the invention was used to treat underarm and leg tissue. A 72 year old man with sagging tissue on the bottom of his upper arms and in his lower legs applied an arm and leg lifting cream comprising of a delivery vehicle of penetrating cream, L-arginine (12.5% w/v), sodium chloride (10% w/v), and magnesium chloride (5% w/v) to his upper arms and lower legs by covering them with cream and rubbing the cream in for five minutes. After about one hour, the sagging tissue was substantially lifted up and firmed. The effect persisted for about seven hours. He continued the treatment of his arms and legs daily for one week. At the end of the week the treatment resulted in youthful looking arms and legs with the sag apparently completely reversed. The effect lasted 11-17 hours.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising an act of:
applying a delivery vehicle comprising a nitric oxide donor to a region of sagging skin for a period of time sufficient to reduce sagging.

2. The method of claim 1, wherein the sagging is determined using viscoelasticity.

3. The method of claim 1, wherein the delivery vehicle is a cream.

4. The method of claim 1, comprising rubbing the delivery vehicle into the region of skin.

5. The method of claim 1, wherein the region of skin is part of one or more of the breast, chin, neck, face, forehead, an arm, a leg, a buttock, and/or an ankle.

6. The method of claim 1, wherein the nitric oxide donor comprises L-arginine.

7. The method of claim 6, wherein the effective concentration of L-arginine is at least 5% by weight/volume of the delivery vehicle.

8. The method of claim 1, wherein the delivery vehicle further comprises one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A, vitamin D, triethanolamine, methylparaben, aloe vera extract, imidazolidinyl urea, propylparaben, PND, or BHA.

9. The method of claim 1, further comprising an act of reapplying the delivery vehicle to the region of skin.

10. The method of claim 9, comprising repeating the act of reapplying the delivery vehicle to the region of skin between 2 and 30 times, inclusively, within a time period of about 30 days.

11. The method of claim 1, wherein the delivery vehicle further comprises a penetrating agent.

12. The method of claim 11, wherein the penetrating agent is present in the delivery vehicle at a concentration at least sufficient to allow the nitric oxide donor to act for at least about 3 hours.

13. The method of claim 11, wherein the penetrating agent comprises an ionic salt.

14. The method of claim 13, wherein the ionic salt comprises one or more of lithium chloride, sodium chloride, potassium chloride, calcium chloride, magnesium chloride, or choline chloride.

15. The method of claim 13, wherein the ionic salt is present at a concentration of at least about 10% by weight.

16. The method of claim 1, wherein the nitric oxide donor comprises one or more of a polysaccharide-bound nitric oxide-nucleophile adduct, a N-nitroso-N-substituted hydroxylamines, a compound containing a sulfhydryl group and a NO donor group, 1,3-(nitrooxymethyl)phenyl-2-hydroxybenzoate, a gel comprising a nitrite salt and an acid, S-nitrosothiols, a nitrite, a 2-hydroxy-2-nitrosohydrazine, a substrate for nitric oxide synthase, a cytokine, an adenosine, bradykinin, calreticulin, bisacodyl, phenolphthalein, or endothelein.

17. The method of claim 1, wherein the delivery vehicle contains a hostile biophysical environment.

18. A method, comprising an act of:
applying a delivery vehicle to a region of skin containing a nitric oxide donor for a period of time sufficient to allow the skin to absorb a sufficient quantity of nitric oxide to produce a smoother surface in the region of skin.

19. The method of claim 18, wherein the delivery vehicle is a cream.

20. The method of claim 18, comprising rubbing the delivery vehicle into the region of skin.

21. The method of claim 18, wherein the delivery vehicle comprises one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A, vitamin D, triethanolamine, methylparaben, aloe vera extract, imidazolidinyl urea, propylparaben, PND, or BHA.

22. The method of claim 18, further comprising an act of reapplying the delivery vehicle to the region of skin.

23. The method of claim 22, comprising repeating the act of reapplying the delivery vehicle to the region of skin after between about 8 hours and about 48 hours after the act of applying the delivery vehicle.

24. The method of claim 18, wherein the nitric oxide donor comprises L-arginine.

25. The method of claim 18, wherein the delivery vehicle further comprises a penetrating agent.

26. The method of claim 25, wherein the penetrating agent comprises an ionic salt.

27. The method of claim 26, wherein the ionic salt comprises one or more of lithium chloride, sodium chloride, potassium chloride, calcium chloride, magnesium chloride, or choline chloride.

28. The method of claim 26, wherein the ionic salt is present at a concentration of at least about 10% by weight.

29. A method, comprising:
administering, to a subject diagnosed as having breast ptosis, a composition comprising a nitric oxide donor.
